(19) United States
(12) Patent Application Publication
(10) Pub. No.: US 2026/0345992 A1
(43) Pub. Date: Nov. 5, 2026

(54) FAIL SAFE INTERATRIAL SHUNT DEVICE

(71) Applicant: UNIVERSITY OF LEICESTER, Leicester (GB)

(72) Inventors: Mohammad Reza Bahmanyar, London (GB); Piyal Samara-Ratna, Leicester (GB); David Adlam, Leicester (GB)

(21) Appl. No.: 19/102,001

(22) PCT Filed: Jul. 13, 2023
(86) PCT No.: PCT/GB2023/051834

§ 371 Date: Feb. 7, 2025

(30) Foreign Application Priority Data
Aug. 9, 2022 (GB) 2211616.4

(51) Int. Cl. A61M 60/165; A61F 2/24; A61F 2/48; A61M 27/00; A61M 60/531

(52) U.S. Cl. CPC A61M 60/165; A61F 2/2427; A61F 2/2469; A61F 2/2476; A61F 2/482; A61M 27/002; A61M 60/531; A61F 2210/0014; A61F 2250/0067

(57) ABSTRACT

An active shunt apparatus includes a fixture attachable to a septum of a patient's heart to form a passage between left and right chambers of the patient's heart. The apparatus also includes a reversibly deformable occluder configured to control a flow of blood through the passage between the left and right chambers, at least one pressure sensor configured to measure a blood pressure associated with one or more portions of the patient's cardiovascular system, and a control unit configured to, based on a signal received from the at least one pressure sensor, cause deformation of the occluder and a corresponding change in the blood pressure.

### TECHNICAL FIELD

The present disclosure relates to an active shunt apparatus and, in particular, to an active shunt apparatus generally comprising a fixture attachable to a septum of a patient's heart, a reversibly deformable occluder, at least one pressure sensor and a control unit. An associated control unit, method and computer program are also disclosed.

### BACKGROUND

Heart failure is a leading cause of premature cardiovascular morbidity and mortality, is costly both for patients' quality of life and health service provision, and has a detrimental impact on GDP. Patients often become destabilised before the onset of acute heart failure, and in combination with severe breathlessness require hospitalisation to be restabilised —a treatment that comes at great cost to a health service. It is well established that acute heart failure episodes are themselves associated with further cardiac injury and a step-wise deterioration in bodily function.

Acute heart failure is characterised by rising left ventricular filling pressure, LVFP. Recently, devices have been developed to monitor pulmonary artery pressure, PAP, a close approximation to LVFP. Whilst PAP monitoring is a significant advance, substantial issues remain: patients may still require hospital admission, indicating a continued need for prevention of acute heart failure admissions; and the clinical resources required to monitor and respond to changes in PAP, by timely action or changes in clinical/medical management in order to prevent acute heart failure, are substantial.

Accordingly, there is a need for further advances in PAP monitoring to realise a paradigm shift in outcomes in acute heart failure.

The listing or discussion of a prior-published document or any background in this specification should not necessarily be taken as an acknowledgement that the document or background is part of the state of the art or is common general knowledge. One or more aspects/examples of the present disclosure may or may not address one or more of the background issues.

### SUMMARY

According to a first aspect, there is provided an active shunt apparatus comprising:
a fixture attachable to a septum of a patient's heart to form a passage between left and right chambers of the patient's heart;
a reversibly deformable occluder configured to control a flow of blood through the passage between the left and right chambers;
at least one pressure sensor configured to measure a blood pressure associated with one or more portions of the patient's cardiovascular system; and
a control unit configured to, based on a signal received from the at least one pressure sensor, cause deformation of the occluder and a corresponding change in the blood pressure.

The control unit may be configured to cause deformation of the occluder when the measured blood pressure is above or below a predefined pressure threshold.

The control unit may be configured to cause deformation of the occluder from a first state to a second state to increase the flow of blood from the left chamber to the right chamber when one or more of the left atrial filling pressure, left-right atrial differential pressure, left ventricular filling pressure and pulmonary artery pressure is above the predefined pressure threshold.

The control unit may be configured to generate a signal to notify a user of the active shunt apparatus that deformation of the occluder from the first state to the second state has occurred.

The control unit may be configured to cause deformation of the occluder to maintain the blood pressure within a predefined pressure range.

The occluder may be configured to undergo continuously variable deformation, from anywhere between a first state allowing a minimum rate of blood flow through the passage and a second state allowing a maximum rate of blood flow through the passage, to maintain the blood pressure within the predefined pressure range.

The occluder may be configured such that the minimum rate of blood flow through the passage is non-zero.

The control unit may be configured to further cause periodic deformation of the occluder to reduce tissue formation within the passage or endothelialisation of the occluder.

The control unit may comprise an actuator connectable to the occluder by a cable, and the actuator may be configured to pull, push or release the cable in response to the signal received from the at least one pressure sensor to cause deformation of the occluder.

The actuator may comprise one or more of an electromechanical system, an electromagnetic system, a microgear system, a microwinch, a motor, a pair of rollers, and a piezoelectric drive mechanism.

The fixture may comprise distal and proximal flange portions configured to contact respective left and right walls of the septum when attached, and one end of the occluder may be attached to the cable such that the occluder abuts the proximal flange portion of the fixture to inhibit the flow of blood when the cable is pushed or released.

The cable may comprise an inner tube axially movable within a stationary outer tube, one end of the outer tube may be attached to a housing of the actuator and the other end of the outer tube may be attached to the distal flange portion of the fixture. The inner tube of the cable may be a helically wound spiral tube and the outer tube of the cable may be a braided polymer tube.

The active shunt apparatus may further comprise a rigid tube having first and second ends, the first end of the rigid tube attached to a first end of the occluder and the second end of the rigid tube attached to the inner tube of the cable. A second end of the occluder may be attached to the inner tube of the cable via a slider configured to slide coaxially within the rigid tube. The inner tube of the cable may comprise an electrically conductive material and may be configured to form an electrical connector of the at least one pressure sensor. The inner tube of the cable may be configured to form a ground connector of the at least one pressure sensor.

The active shunt apparatus may further comprise a piston housed within a cylinder, a first end of the piston attached to the occluder and a second end of the piston attached to the cable. The first end of the piston may be attached to the occluder via a bearing configured to inhibit rotational motion of the cylinder. The cylinder may be attached to the fixture via one or more collapsible arms.

The occluder may be attached to, or formed as an extension of, the fixture. The fixture and occluder may be sufficiently collapsible to enable endovascular delivery thereof via a catheter. The fixture may have a collapsible wire mesh structure and the occluder may have a collapsible wire mesh structure or a collapsible spiral spring structure.

The collapsible wire mesh structure or collapsible spiral spring structure may be formed from a super-elastic material. The super-elastic material may comprise a shape memory alloy such as nickel titanium.

At least part of the collapsible wire mesh structure or collapsible spiral spring structure may comprise a polymer coating or membrane. The polymer coating or membrane may be formed from one or more of polyurethane, silicone and polyethylene terephthalate, optionally impregnated with an eluting agent.

The septum may be an atrial septum, the left chamber may be a left atrium and the right chamber may be a right atrium; or the septum may be a ventricular septum, the left chamber may be a left ventricle and the right chamber may be a right ventricle.

The blood pressure may be a differential blood pressure between the left and right chambers of the patient's heart.

The active shunt apparatus may comprise a plurality of pressure sensors configured to measure one or more portions of the patient's cardiovascular system.

According to a second aspect, there is provided a control unit of an active shunt apparatus,
the active shunt apparatus further comprising a fixture attachable to a septum of a patient's heart to form a passage between left and right chambers of the patient's heart; a reversibly deformable occluder configured to control a flow of blood through the passage between the left and right chambers; and at least one pressure sensor configured to measure a blood pressure associated with one or more portions of the patient's cardiovascular system,
   the control unit configured to:
   receive a signal from the at least one pressure sensor indicative of the measured blood pressure; and
   cause deformation of the occluder and a corresponding change in the blood pressure.

The control unit may comprise at least one processor and at least one memory including computer program code, the at least one memory and computer program code configured to, with the at least one processor, cause the control unit to receive the signal from the at least one pressure sensor and cause deformation of the occluder.

According to a third aspect, there is provided a method of controlling blood pressure comprising:
attaching a fixture to a septum of a patient's heart to form a passage between left and right chambers of the patient's heart;
providing a reversibly deformable occluder configured to control a flow of blood through the passage between the left and right chambers;
measuring a blood pressure associated with one or more portions of the patient's cardiovascular system using at least one pressure sensor; and
causing deformation of the occluder and a corresponding change in the blood pressure, based on a signal received from the at least one pressure sensor, using a control unit.

According to a fourth aspect, there is provided a method of controlling blood pressure using a control unit of an active shunt apparatus,
the active shunt apparatus further comprising a fixture attachable to a septum of a patient's heart to form a passage between left and right chambers of the patient's heart; a reversibly deformable occluder configured to control a flow of blood through the passage between the left and right chambers; and at least one pressure sensor configured to measure a blood pressure associated with one or more portions of the patient's cardiovascular system,
   the method comprising:
   receiving, at the control unit, a signal from the at least one pressure sensor indicative of the measured blood pressure; and
   causing, by the control unit, deformation of the occluder and a corresponding change in the blood pressure.

According to a fifth aspect, there is provided a computer program comprising computer code configured to perform the method of the fourth aspect.

The computer program may comprise one or more computational algorithms.

The computational algorithms may comprise one or more of a proportional-integral-derivative controller, a (neuro) fuzzy controller, an expert system and an artificial intelligence-based controller.

According to a sixth aspect, there is provided an apparatus as substantially described herein with reference to, and as illustrated by, the accompanying drawings.

The optional features described in relation to the active shunt apparatus of the first aspect are also applicable to the control unit of the second aspect, the method of the third aspect, the method of the fourth aspect, and/or the computer program of the fifth aspect where compatible.

The steps of any method disclosed herein do not have to be performed in the exact order disclosed, unless explicitly stated or understood by the skilled person.

Corresponding computer programs for implementing one or more steps of the methods disclosed herein are also within the present disclosure and are encompassed by one or more of the described examples.

One or more of the computer programs may, when run on a computer, cause the computer to configure any apparatus, including a battery, circuit, controller, or device disclosed herein or perform any method disclosed herein. One or more of the computer programs may be software implementations, and the computer may be considered as any appropriate hardware, including a digital signal processor, a microcontroller, and an implementation in read only memory (ROM), erasable programmable read only memory (EPROM) or electronically erasable programmable read only memory (EEPROM), as non-limiting examples. The software may be an assembly program.

One or more of the computer programs may be provided on a computer readable medium, which may be a physical computer readable medium such as a disc or a memory device, or may be embodied as a transient signal. Such a transient signal may be a network download, including an internet download.

The present disclosure includes one or more corresponding aspects, examples or features in isolation or in various combinations whether or not specifically stated (including claimed) in that combination or in isolation. Corresponding means for performing one or more of the discussed functions are also within the present disclosure.

Throughout the present specification, descriptors relating to position, orientation or movement such as “left”, “right”, “up”, “down”, “horizontal” and “vertical”, as well as any adjective and adverb derivatives thereof, are used in the sense of the position, orientation or movement of the apparatus as presented in the drawings. However, except by way of reference to structures of a patient's heart, such descriptors are not intended to be in any way limiting to an intended use of the described or claimed invention.

The above summary is intended to be merely exemplary and non-limiting.

### BRIEF DESCRIPTION OF THE FIGURES

A description is now given, by way of example only, with reference to the accompanying schematic drawings, in which:

FIG. 1 shows an illustration of an active shunt apparatus according to an example.

FIGS. 2a-b show illustrations of the example active shunt apparatus of FIG. 1 in sealed and unsealed configurations respectively.

FIG. 3 shows an illustration of an active shunt apparatus according to another example.

FIG. 4 shows an illustration of the active shunt apparatus of FIG. 3 in an unsealed configuration.

FIGS. 5a-d show further illustrations of the active shunt apparatus of FIGS. 3 and 4.

FIG. 6 shows an illustration of an active shunt apparatus according to another example.

FIG. 7 shows in schematic form a control unit of an active shunt apparatus according to an example.

FIGS. 8a-d shown screenshots of an example active shunt apparatus under testing.

FIG. 9 shows a method of controlling blood pressure.

FIG. 10 shows a method of controlling blood pressure using a control unit of an active shunt apparatus.

FIGS. 11a-j show an assembly flow for an active shunt apparatus according to an example.

FIG. 12 shows an illustration of an example active shunt apparatus that relates to the example active shunt apparatus shown in FIG. 6.

### DETAILED DESCRIPTION

### General overview

Recently, it has been hypothesized that the creation of a left-to-right interatrial shunt to decompress the left atrium could be effective for alleviating symptoms in heart failure (HF) patients [1], and shown (REDUCE LAP-HF I trial) that “a transcatheter interatrial shunt device (IASD, Corvia Medical) was associated with lower pulmonary capillary wedge pressure, fewer symptoms, and greater quality of life and exercise capacity in patients with heart failure” [2].

However, the interatrial shunt device (IASD) is a passive device with a fixed diameter interatrial opening, through which the left atrium is continuously decompressed, even when the left ventricular filling pressure (LVFP) is not elevated. This is problematic as it is well established that a fixed interatrial shunt will, over time, lead to increased right heart blood flows and secondary pulmonary hypertension (akin to Eisenmenger's syndrome) [3,4]. Moreover, a fixed interatrial shunt is not capable of maintaining LVFP within an optimal range.

An active pressure responsive leakage control (i.e., an active shunt) is desirable to maintain the filling pressure within a desired range and is an unmet clinical need. Such a device is clinically viable only if it can be delivered endovascularly via routes familiar to interventionists (like a pacemaker procedure). This means that the whole leakage control system should be collapsible to fit into a delivery catheter.

According to examples of the present disclosure, an interatrial (active) shunt device is a leakage controllable flexible valve system implanted on the heart septum (the wall separating the left and right heart atria) and is deployable endovascularly and connectable to a control unit via a flexible cable. The control unit may be implanted under a patient's skin, such as near the collar bone, like a pacemaker. The valve system may be equipped with miniature pressure sensors used to monitor left and/or right atrial pressures continuously. The pressure sensor readings are communicated to the control unit and used to adjust the leakage rate of the valve, thereby relieving abnormally high filling pressure in the left atrium. By relieving the pressure in the left atrium (i.e., by transiently allowing blood to pass to the lower pressure right atrium), left atrial pressure rising beyond a predetermined level is prevented. In turn, this prevents progression to rising pulmonary pressure, pulmonary oedema and an acute heart failure admission.

Apart from causing automatic pressure adjustment, the control unit may be capable of wirelessly transmitting an operation log to clinicians to allow a clinical team to respond to and modify a patients' clinical management to restore clinical equilibrium. At such a time, the device will function to close the shunt and return to its quiescent monitoring status. Alternatively, the device can work in closed loop mode to maintain the left atrium filling pressure within a pre-set safe range.

Additionally, the control unit may be equipped with wireless interfaces enabling it to be programmed by an external device outside the patient.

### Active Shunt Apparatus

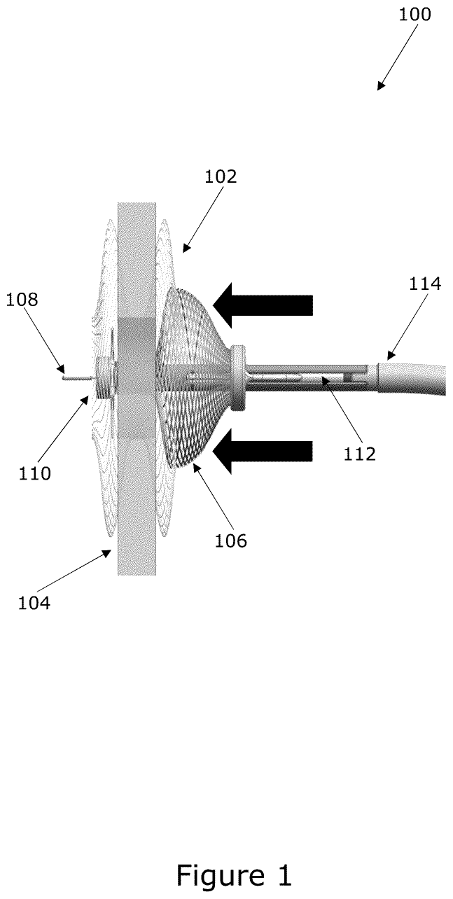

FIG. 1 shows an illustration of an active shunt apparatus 100 according to an example, which may be characterised as providing leakage control by a conforming occluder. The active shunt apparatus 100 comprises a fixture 102, which may be termed a shunt, attachable to a septum 104 of a patient's heart to form a passage between left and right chambers of the patient's heart, a reversibly deformable occluder 106 configured to control a flow of blood through the passage between the left and right chambers, at least one pressure sensor 108 configured to measure a blood pressure associated with one or more portions of the patient's cardiovascular system, and a control unit (not shown) configured to, based on a signal received from the at least one pressure sensor 108, cause deformation of the occluder 106 and a corresponding change in the blood pressure.

The example active shunt apparatus 100 further comprises fixing means 110 to fix the fixture 102 to the reversibly deformable occluder 106, a flexible cable 112, and an outer polymer sheath 114. Some or all of these features may however be dispensed with in other examples.

The septum 104 may be an atrial septum, the left chamber may be a left atrium and the right chamber may be a right atrium; or the septum 104 may be a ventricular septum, the left chamber may be a left ventricle and the right chamber may be a right ventricle. The blood pressure may be a differential blood pressure between the left and right chambers of the patient's heart. The active shunt apparatus 100 may comprise a plurality of pressure sensors configured to measure one or more portions of the patient's cardiovascular system. The fixture 102 and the reversibly deformable occluder 106 may be termed a valve system.

The valve system may comprise a Nitinol wire wound (or laser cut) shunt and a wire wound (or laser cut) reversibly deformable occluder 106 made from super-elastic Nitinol. The reversibly deformable occluder 106 may contain a Dacron membrane embedded into a thin elastomer (such as polyurethane or silicone) to provide sealing to the shunt. Alternatively, at least part of the reversibly deformable occluder 106 surface, for example the surface adjacent to the fixture 102, may be coated with an elastomer suitable for permanent implantation, for example NuSil™ MED-6605, to provide a sealing surface non-permeable to blood.

The reversibly deformable occluder 106 is fixed to the shunt on the left side of the septum 104 via the fixing means 108 and is connected, via the flexible cable 112, to the control unit. The flexible cable 112 runs through the outer polymer sheath 114 and is connected to the right-hand side (as viewed according to FIG. 1) of the reversibly deformable occluder 106. The control unit contains an electromechanical system capable of pulling and pushing (or releasing under tension) the flexible cable 112, thereby deforming the reversibly deformable occluder 106 (as illustrated by the block-filled arrows) and therefore controlling the leakage of blood from the left atrium to the right atrium. That is, to form a seal the reversibly deformable occluder 106 is pressed against the shunt. Two pressure sensors (not shown) monitor the right and left atria pressures, and an electronic system (not shown) determines the extent to which the cable is pulled or released.

In use the shunt typically becomes endothelialised-i.e., endothelial tissue forms on the shunt. To provide an enhanced sealing effect, the shunt may be embedded in a polymer structure before use.

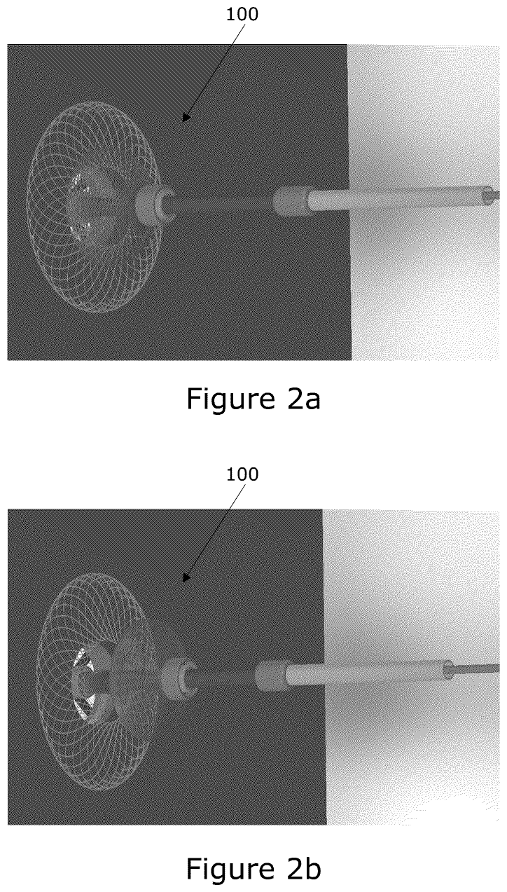

FIGS. 2a-b show illustrations of the example active shunt apparatus 100 in sealed (valve closed) and unsealed (valve open) configurations respectively.

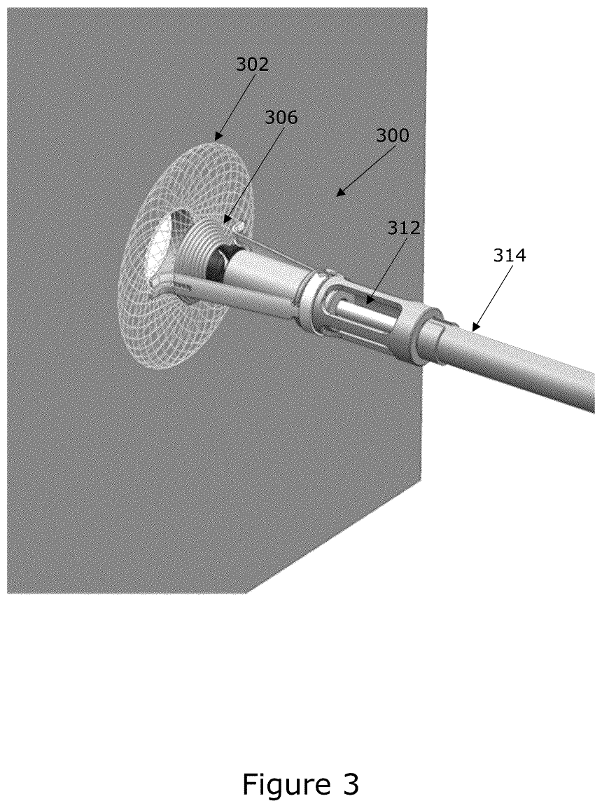

FIG. 3 shows an illustration of an active shunt apparatus 300 according to another example, which may be characterised as providing leakage control by a spring (conical wire) occluder. The active shunt apparatus 300 comprises a fixture 302, a reversibly deformable occluder 306, at least one pressure sensor (not shown), and a control unit (not shown) that function as described with reference to the active shunt apparatus shown in FIGS. 1 and 2a-b. The active shunt apparatus 300 further comprises, as optional features, a flexible cable 312, and an outer polymer sheath 314.

The active shunt apparatus 300 is shown in FIG. 3 in a sealed (valve closed) configuration.

The concept of leakage control in this example is similar to the concept of the previous example, but is achieved via a conically wound spring, which may be made from super-elastic Nitinol. As with the previous example, the conically wound spring may be coated with a polymer layer before use to provide for an enhanced sealing effect.

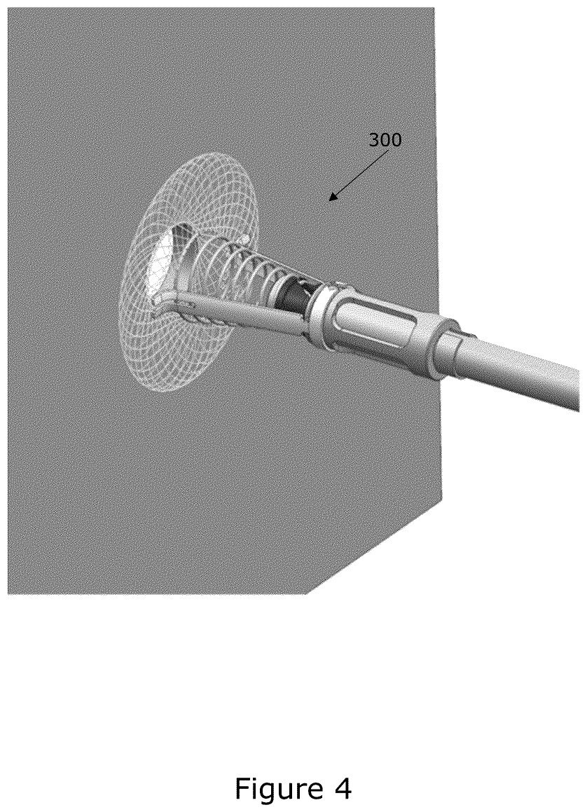

FIG. 4 shows an illustration of the active shunt apparatus 300 in an unsealed (valve open) configuration.

Accordingly, the control unit may be configured to cause deformation of the occluder 106 when the measured blood pressure is above or below a predefined pressure threshold. For example, the control unit may be configured to cause deformation of the occluder 106 from a first state to a second state to increase the flow of blood from the left chamber to the right chamber when one or more of the left atrial filling pressure, left-right atrial differential pressure, left ventricular filling pressure and pulmonary artery pressure is above the predefined pressure threshold. In this example, the control unit may be configured to generate a signal to notify a user of the active shunt apparatus 100 that deformation of the occluder 106 from the first state to the second state has occurred.

Additionally or alternatively, the control unit may be configured to cause deformation of the occluder 106 to maintain the blood pressure within a predefined pressure range (i.e. closed-loop mode). In this example, the occluder 106 may be configured to undergo continuously variable deformation, from anywhere between a first state allowing a minimum rate of blood flow through the passage and a second state allowing a maximum rate of blood flow through the passage, to maintain the blood pressure within the predefined pressure range. The occluder 106 may be configured such that the minimum rate of blood flow through the passage is non-zero.

The collapsible wire mesh structure or collapsible spiral spring structure may be formed from a super-elastic material. The super-elastic material may comprise a shape memory alloy such as nickel titanium.

At least part of the collapsible wire mesh structure or collapsible spiral spring structure may comprise a polymer coating or membrane. The polymer coating or membrane may be formed from one or more of polyurethane, silicone and polyethylene terephthalate, optionally impregnated with an eluting agent.

The following discussion serves to support the above examples and to introduce additional optional features.

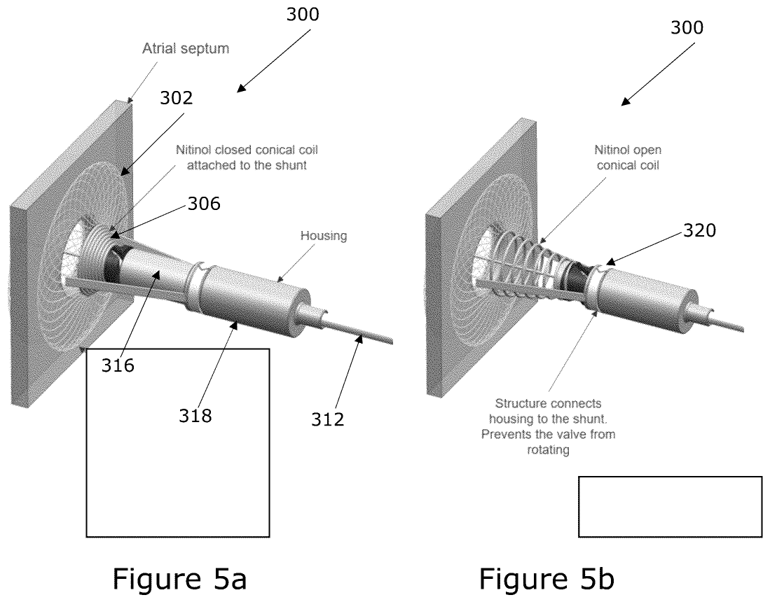

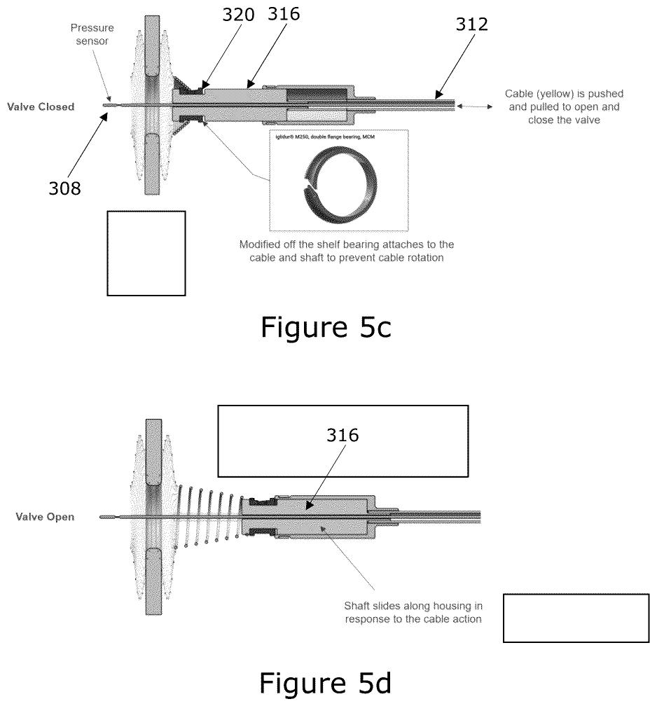

FIGS. 5a-d show further illustrations of the active shunt apparatus 300. Whereas FIGS. 5a and 5c show the active shunt apparatus 300 in a sealed (valve closed) configuration, FIGS. 5b and 5d show the active shunt apparatus 300 in an unsealed (valve open) configuration.

The conical wire device concept uses a conical wire spring 306 to seal and offload pressure between the left and right atria. When pushed (i.e., closed) against the shunt, the contacting spring wires form a seal to minimise leakage. When pulled away from the shunt, an open pathway is created to offload pressure. See FIGS. 5c and 5d, respectively, in particular.

To help the sealing performance the wire may be coated, e.g. with silicone. The conical wire may be either a wire extension from a Nitinol wire shunt or a separate element attached to the shunt, e.g. by bonding, braising or welding.

The smaller cross-section end of the conical wire spring 306 is connected to a piston 316 housed within a cylinder 318. An intermediate bearing 320 provided between the conical wire spring 306 and the piston 316 prevents rotation of the cylinder housing 318 affecting the spring's shape (i.e., the intermediate bearing 320 connects the cylinder housing 318 to the shunt 302 to prevent the valve from rotating). A double flange bearing, MCM, such as the iglidur® M250, may be used for this purpose.

The piston 316 is pulled/pushed by a cable 312 attached to the implanted drive system to open and close the valve formed by the conical wire spring 306. The piston 316 slides within the cylinder housing 318 in response to actuation of the cable 312.

A pressure sensing wire (not shown) is provided through the centre of the piston 316 to enable pressure monitoring by the pressure sensor 308 of the left atrial chamber. Alternative locations of the pressure sensing wire (e.g., off-centre of the piston 316) can also be used where compatible.

To improve resistance against lateral blood flow movement the active shunt apparatus 300 may have multiple Nitinol arms spanning between the cylinder 316 and the shunt 302. This increases radial stiffness of the device. In delivery the super-elastic Nitinol arms and spring wire 306 collapse to the same diameter as the piston 316. The diameter of the cylinder housing 318 is sized to fit inside an appropriately sized delivery catheter. Shapes other than cylindrical, such as frusto-conical, may also be used for the housing 318 and/or the piston 316 where compatible.

The example active shunt apparatus of the present disclosure may enable an immediate, or near immediate, response to protect the heart against an acute rise in LVFP. As such, these devices have considerable potential to prevent a downward spiral towards acute heart failure. These devices may also contribute to substantially reduced health care costs by further reducing acute heart failure admissions and eliminating the need for a dedicated team to monitor and respond to changes in pulmonary artery pressure signals.

Benefits of the spring (conical wire) occluder design may include one or more of:

The wire spring requires a relatively low actuation force to open and close. This can improve battery life of the drive system.

The relatively low actuation force can allow the wires to open and close more frequently to prevent clotting or endothelialisation from occurring.

The area of the opening created by the spring is relatively large, which may help with pressure offloading and/or reduce damage to blood cells.

The device configuration reduces the volume (‘dead volume’) for clotting or endothelialisation to occur.

More generally:

The design provides an effective seal when compressed. This effectiveness can be enhanced by coating the wires with an anti-coagulant coating or ductile sealing material, e.g. silicone.

The closed area formed by the spring surface leaves minimal dead volume space, thereby reducing the likelihood of blood stagnating and clotting.

The size and tapered shape of the conical spring facilitate insertion into a delivery catheter. The proximal end of the conical spring is sized for consistency with the size of a septum aperture.

In an unsealed, open configuration the wire creates a relatively large opening area that allows blood to pass through relatively freely, thus providing for effective pressure offloading. A thin diameter wire (e.g., 120-250 μm) may have a minimal effect on altering blood flow.

The extent to which the spring can be opened is controllable to provide variable levels of pressure offloading.

The actuation forces required to open and close the conical spring are relatively low, which may increase drive system operational lifetime (since less power is required to actuate the conical spring). Lower operational power also provides options to actuate the device more frequently to prevent clotting or tissue growth.

Preferably, the conical wire is made from a super-elastic material. This allows the wire to be stretched to fit inside a delivery catheter and unfurl to a fully formed conical shape when deployed. The most common materials in this respect are shape memory alloys such as Nitinol.

Accordingly, the active shunt apparatus 300 may further comprise a piston 316 housed within a cylinder 316, a first end of the piston attached to the occluder 300 and a second end of the piston 316 attached to the cable 312. The first end of the piston 316 may be attached to the occluder 606 via a bearing 320 configured to inhibit rotational motion of the cylinder 316. The cylinder 316 may be attached to the fixture 310 via one or more collapsible arms.

The occluder 306 may be attached to, or formed as an extension of, the fixture 302. The fixture 302 and occluder 306 may be sufficiently collapsible to enable endovascular delivery thereof via a catheter. The fixture 302 may have a collapsible wire mesh structure and the occluder 306 may have a collapsible wire mesh structure or a collapsible spiral spring structure.

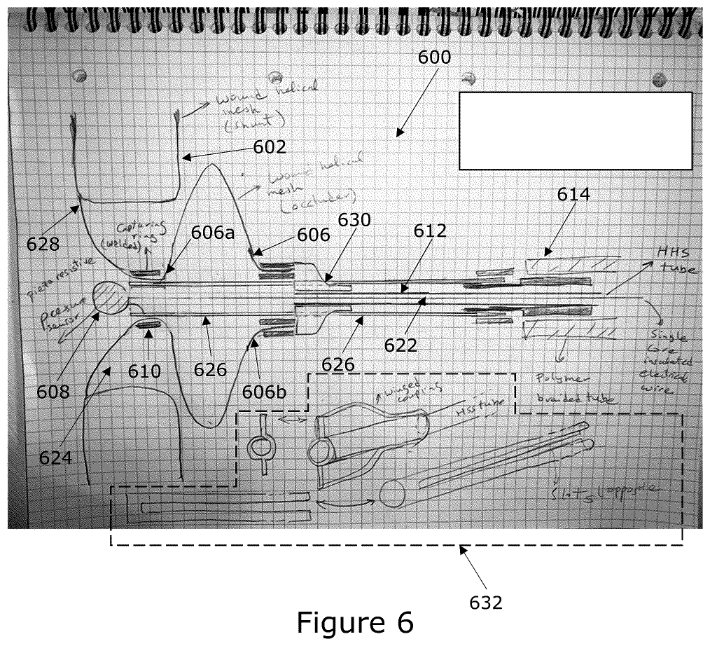

FIG. 6 shows an illustration of an active shunt apparatus 600 according to another example. The active shunt apparatus 600 comprises a fixture 602 attachable to a septum (not shown) of a patient's heart to form a passage between left and right chambers of the patient's heart, a reversibly deformable occluder 606, at least one pressure sensor 608, and a control unit (not shown) that function as described with reference to the active shunt apparatus shown in FIGS. 1-5d. The active shunt apparatus 600 further comprises, as optional features, fixing means 610, a flexible cable (inner flexible cable or control cable) 612, and an outer polymer sheath 614; one or more other features of the active shunt apparatus described with reference to FIGS. 1-5 may also be included where compatible.

The fixture 602 and the reversibly deformable occluder 606 may each be a wound mesh. The at least one pressure sensor 608 may be a piezoelectric, piezoresistive or capacitive pressure sensor, which may be connected to the control unit via a single core insulated electrical wire 622. The reversibly deformable occluder 606 may be connected to a rigid tube 626.

The fixing means 610 in this example is a capturing ring formed by welding. The flexible cable 612 may be a stranded wire tube. The outer polymer sheath 614 may be a polymer braided tube.

In terms of the overall function, the active shunt apparatus 600 consists of two main parts: the wire wound or laser cut nitinol double flange (the fixture or shunt 602) and the wire wound deformable nitinol mesh (the occluder 606). Both of these parts may be made from other materials. Both of these parts are preferably of a super-elastic nature and collapsible for stowing into a delivery tube (catheter).

The occluder 606 is disposed on the septum and provides a fixing thereto. The occluder 606 also provides an opening 624 (the space co-located with the capturing ring 610) between the left and right atria. The occluder 606 is fixed at one end, the distal end, 606a to the rigid tube 626 by the capturing ring 610, which may be welded, as indicated, or soldered/braised. The capturing ring 610 may alternatively comprise occluder wires for fixing to the rigid tube 626.

The distal end 606a of the occluder 606 is also fixed to the left flange 628 of the shunt 602, either by capturing the wires extending from the left flange or by welding a spider super-elastic structure to the left flange 628.

In this arrangement, the left flange 628, the leftmost (distal) part of the occluder 606 and the rigid tube 626 are all fixed together. The rigid tube 626 is coupled to the outer sheath 614 of the control cable 612 that extends to the exterior of the patient. The arrangement of the outer sheath 614, the rigid tube 626, the leftmost part of the occluder 606 and the left flange 628 of the shunt 602 form a stationary section. This is beneficial since, during the deformation of the occluder 606 via the inner flexible tube 612, the applied force on the septum should be minimised (the septum should not move as a result of operating the apparatus).

The proximal end, 606b of the occluder 606 is coupled to a slider 630 that is connected to the inner flexible tube 612 and can slide coaxially in the rigid tube 626. See also the exploded view arrangement 632. The coupling may be a winged coupling. This mechanism allows for pulling/pushing the proximal end 606b of the occluder 606 and deforming the occluder 606 while the outer sheath 614 and therefore septum is stationary. One end of the outer sheath 614 that extends to the exterior of patient is rigidly connected to the control unit, so once implanted inside a control housing, the outer sheath 614 and the connected parts remain stationary upon apparatus operation.

The inner flexible tube 612 is configured to communicate a pulling or pushing action to the occluder 602 and is connected to an electromechanical mechanism (not shown) controlled by electronics inside the control unit. The inner flexible tube 612 may be a helically wound tube (e.g., a helical hollow strand, HHS, tube by Fort Wayne) made from stainless steel or other compatible material. The inner flexible tube 612 is hollow to allow for the passage of the single core insulated electrical wire 622 that connects to the at least one pressure sensor 608. Other electrical wire arrangements, such as fine insulated electric wires, may also be used.

The at least one pressure sensor 608 faces the left atrium and is used to measure the left atrial pressure, preferably in real-time. The inner metallic tube structure (the inner flexible tube 612) may be used as a ground connection for any electrical signal, e.g., signals from the at least one pressure sensor 608. A second pressure sensor (not shown) may be disposed on the right side of the occluder 606 to measure the right atrial pressure, again preferably in real time. Both left and right atrial pressure signals may be processed by the control unit to control blood flow from the left atrium to the right atrium. The electrical connection to the second pressure sensor may also pass through the inner tube, as described for the at least one pressure sensor 608.

The leakage control (valve) function in this example is based on the extent to which the occluder 606 is deformed with respect to the rim of the shunt hole on the septum. Once the shunt 602 is endothelialised, the rim of the shunt hole on the right-hand side (i.e., the side facing the occluder 606) provides a surface against which blood leakage can be controlled.

The occluder 606 is preferably a mesh structure, and the side of the occluder 602 that faces the opening 624 is preferably coated with a sealing layer comprising an elastomer, thereby allowing for deformation of a surface that is impermeable (or is minimally permeable) to blood. Medical grade silicones (e.g. MED-6605) and polyurethane compositions are suitable candidates. One way of coating the occluder 606 is by dip coating in an elastomeric solution of suitable viscosity, followed by a curing step to form an elastic solid. The elastomeric solution may be coated onto a thin polymer layer that itself is pre-deposited onto the surface of the occluder 606.

The two main parts of the device, the shunt 602 and the occluder 606 described above, may be coupled together and delivered as a single device together with the inner flexible tube 612.

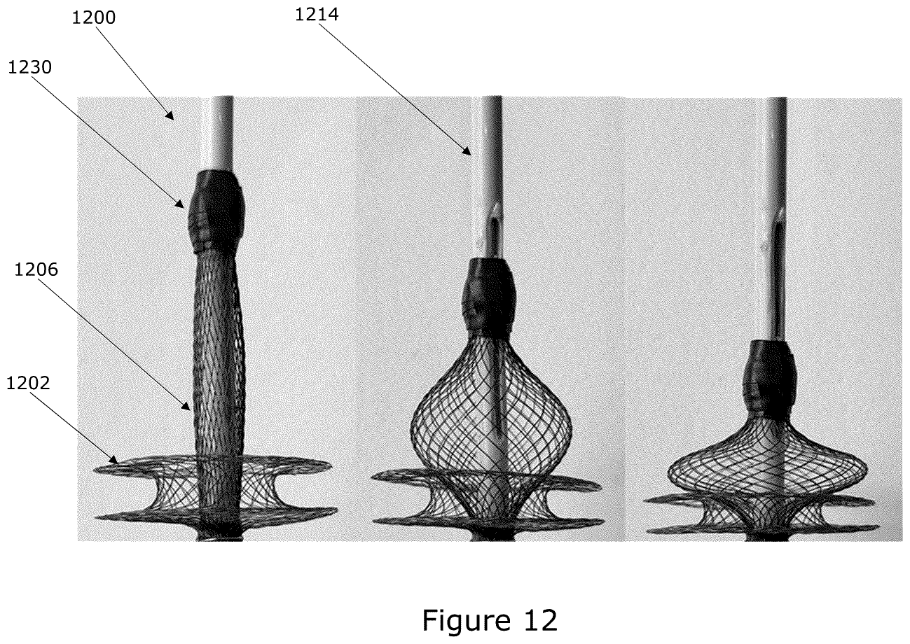

FIG. 12 shows an illustration of an example active shunt apparatus 1200 that relates to the example active shunt apparatus shown in FIG. 6. In this related example, which shows the shunt 1202 and occluder 1206 at various stages of deformation, the shunt 1202 and the occluder 1206 are wound and formed from the same wire strands so as to provide a single unit. Furthermore, fixation to the rigid tube and the outer sheath 1214, as well as to the slider 1230 and the inner tube, is as described for the example active shunt apparatus shown in FIG. 6. Note that the reference signs to the rigid tube and the inner tube of the example of FIG. 12 have been omitted for clarity.

Returning to FIG. 6, alternatively, the shunt 602 and the occluder 606 may be delivered separately. In this example, it is envisaged that the shunt 602 is disposed on the septum first via the procedure described above. The occluder/cable structure is then delivered and fixed to the left flange 628 of the shunt 602 (i.e., the side facing the left atrium) by means of a grabber or a screw mechanism. This method of delivery may be clinically beneficial since it allows for different delivery routes for each part (e.g., femoral as well as subclavian veins).

The control unit may be configured to further cause periodic deformation of the occluder 606 to reduce tissue formation within the passage or endothelialisation of the occluder 606.

The control unit may comprise an actuator connectable to the occluder 606 by a cable, and wherein the actuator is configured to pull, push or release the cable in response to the signal received from the at least one pressure sensor to cause deformation of the occluder 606.

The actuator may comprise one or more of an electromechanical system, an electromagnetic system, a microgear system, a microwinch, a motor, a pair of rollers, and a piezoelectric drive mechanism.

The fixture 610 may comprise distal and proximal flange portions configured to contact respective left and right walls of the septum when attached, and wherein one end of the occluder 606 is attached to the cable such that the occluder 606 abuts the proximal flange portion of the fixture 610 to inhibit the flow of blood when the cable is pushed or released.

The cable may comprise an inner tube 612 axially movable within a stationary outer tube 614, and wherein one end of the outer tube 614 is attached to a housing of the actuator and the other end of the outer tube is attached to the distal flange portion of the fixture 610.

The active shunt apparatus 600 may further comprise a rigid tube 626 having first and second ends, the first end of the rigid tube 626 attached to a first end 606a of the occluder 606 and the second end of the rigid tube 626 attached to the inner tube 612 of the cable. A second end 606b of the occluder 606 may be attached to the inner tube 612 of the cable via a slider 630 configured to slide co-axially within the rigid tube 626. The inner tube 612 of the cable may comprise an electrically conductive material 622 and is configured to form an electrical connector of the at least one pressure sensor.

The inner tube 612 of the cable may be a helically wound spiral tube and the outer tube 614 of the cable may be a braided polymer tube. The inner tube 612 of the cable may be configured to form a ground connector of the at least one pressure sensor 608. In such a configuration, other terminals of the at least one pressure sensor 608 and the second pressure sensor may be connected to the control unit via insulated wires passed through the flexible inner tube 612.

### Control Unit

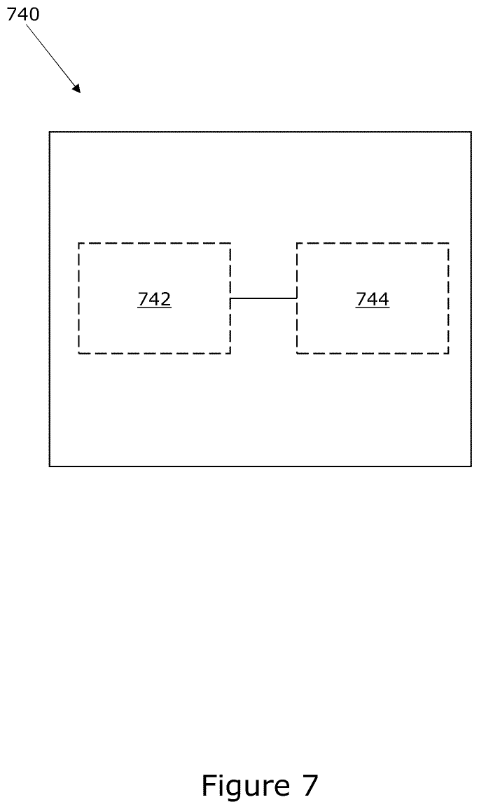

FIG. 7 shows in schematic form a control unit 740 of an active shunt apparatus according to an example, the active shunt apparatus further comprising a fixture attachable to a septum of a patient's heart to form a passage between left and right chambers of the patient's heart; a reversibly deformable occluder configured to control a flow of blood through the passage between the left and right chambers; and at least one pressure sensor configured to measure a blood pressure associated with one or more portions of the patient's cardiovascular system.

The control unit 740 is configured to: receive a signal from the at least one pressure sensor indicative of the measured blood pressure; and cause deformation of the occluder and a corresponding change in the blood pressure.

The control unit may comprise at least one processor 742 and at least one memory 744 including computer program code (as illustrated by the dashed boundaries in FIG. 7), the at least one memory 744 and computer program code configured to, with the at least one processor 742, cause the control unit 740 to receive the signal from the at least one pressure sensor and cause deformation of the occluder.

### Supplementary Data

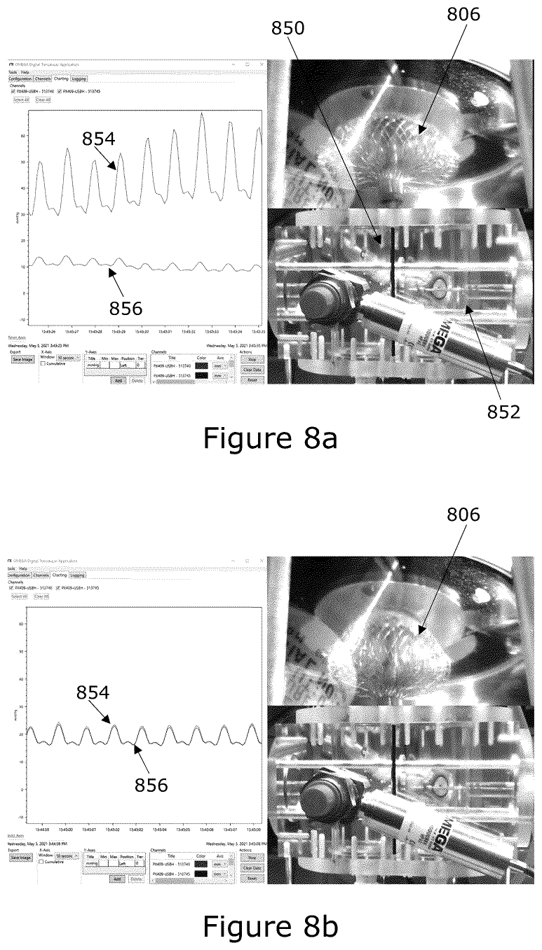

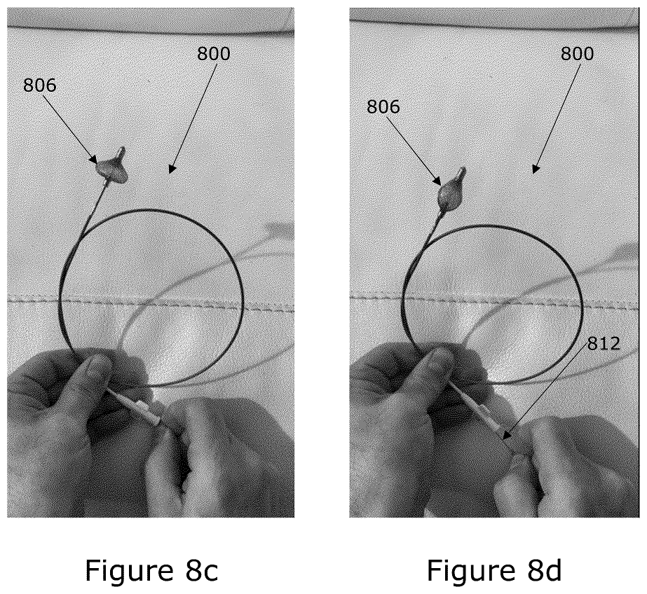

FIGS. 8a-d shown screenshots of an example active shunt apparatus under testing. Specifically, FIG. 8a shows an active shunt apparatus in a closed configuration about an artificial atrial septum opening. In this configuration, the occluder 806 maintains a higher fluid pressure in the left-hand side chamber 850 relative to the fluid pressure in the right-hand side chamber 850, as monitored by respective sensors. See traces 854 and 856 respectively.

FIG. 8b shows the active shunt apparatus an open configuration about the artificial atrial septum opening. See in particular the altered deformation of the occluder 806. This configuration allows fluid pressure to equilibrate between the left-hand side and right-hand side chambers, as evidenced by the matching traces 854 and 856.

FIGS. 8c and 8d show an active shunt apparatus 800 without a control unit. Manual actuation of the flexible cable 812 causes deformation of the occluder 806 in a manner than is consistent with transitioning from a closed (see FIG. 8c) to an open (see FIG. 8d) configuration.

### Methods

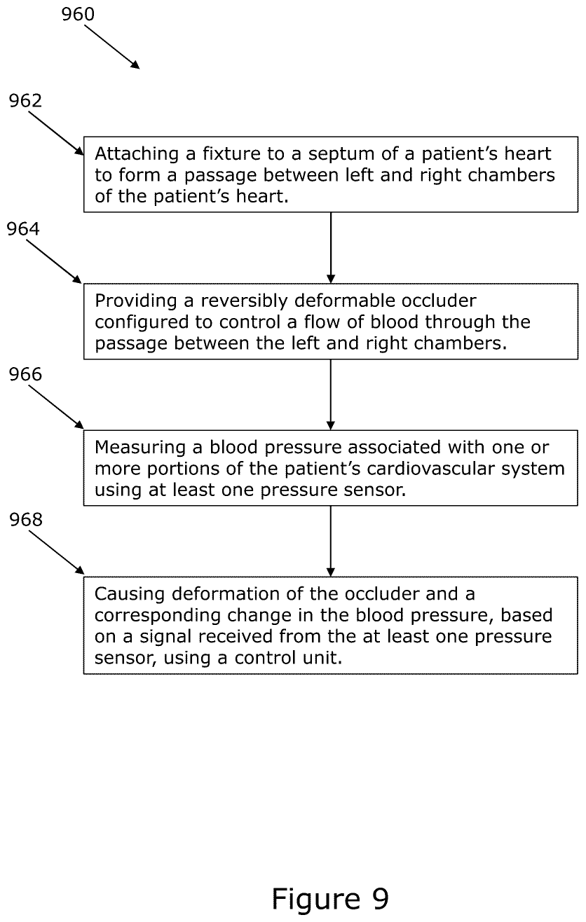

FIG. 9 shows a method 960 of controlling blood pressure. The method 960 comprises: attaching 962 a fixture to a septum of a patient's heart to form a passage between left and right chambers of the patient's heart; providing 964 a reversibly deformable occluder configured to control a flow of blood through the passage between the left and right chambers; measuring 966 a blood pressure associated with one or more portions of the patient's cardiovascular system using at least one pressure sensor; and causing 968 deformation of the occluder and a corresponding change in the blood pressure, based on a signal received from the at least one pressure sensor, using a control unit.

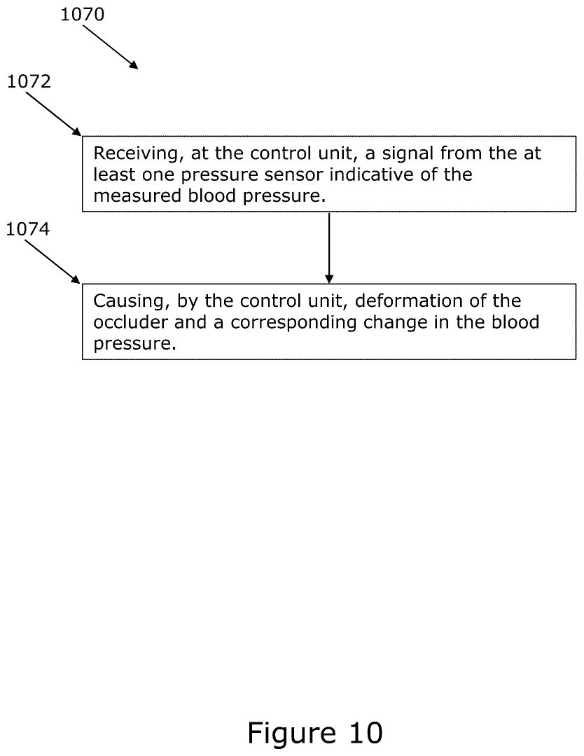

FIG. 10 shows a method 1070 of controlling blood pressure using a control unit of an active shunt apparatus, the active shunt apparatus further comprising a fixture attachable to a septum of a patient's heart to form a passage between left and right chambers of the patient's heart; a reversibly deformable occluder configured to control a flow of blood through the passage between the left and right chambers; and at least one pressure sensor configured to measure a blood pressure associated with one or more portions of the patient's cardiovascular system. The method 1070 comprises: receiving 1072, at the control unit, a signal from the at least one pressure sensor indicative of the measured blood pressure; and causing, by the control unit, deformation of the occluder and a corresponding change in the blood pressure.

### Assembly Flow & Device Delivery

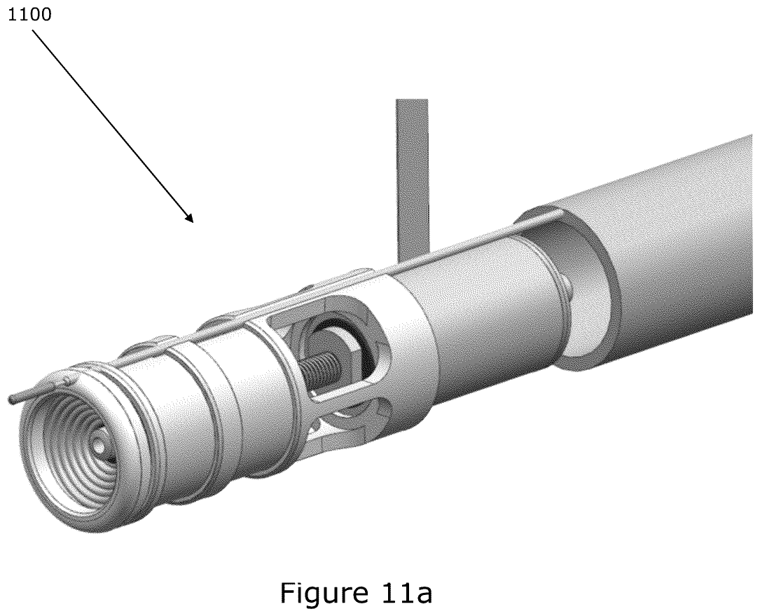

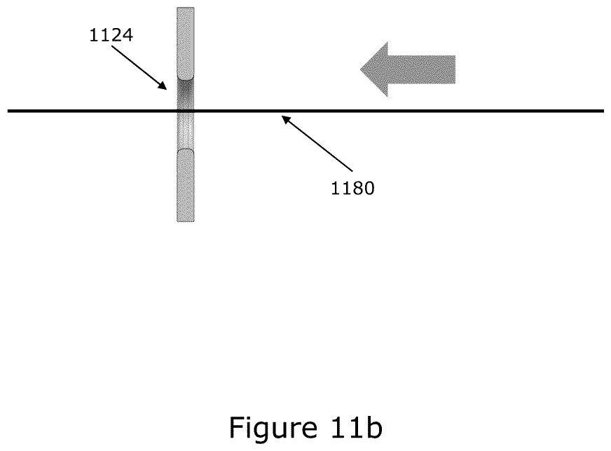

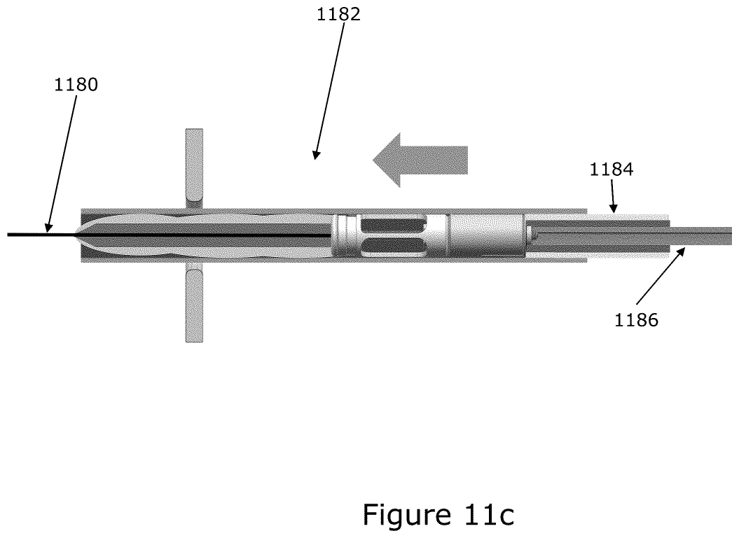

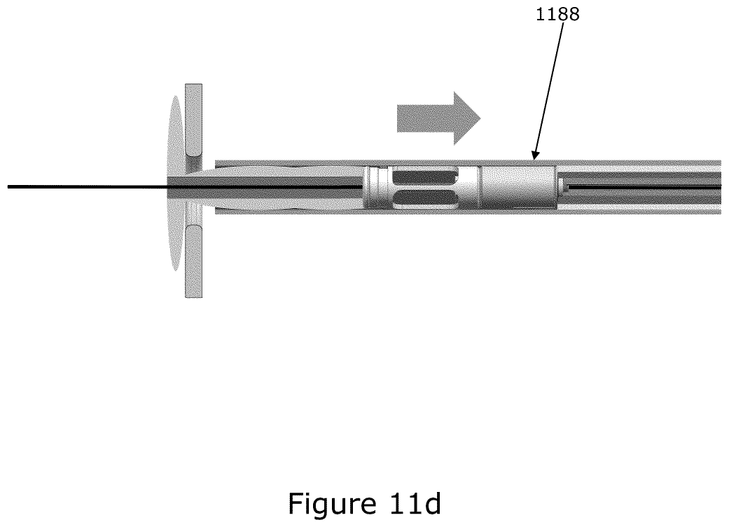

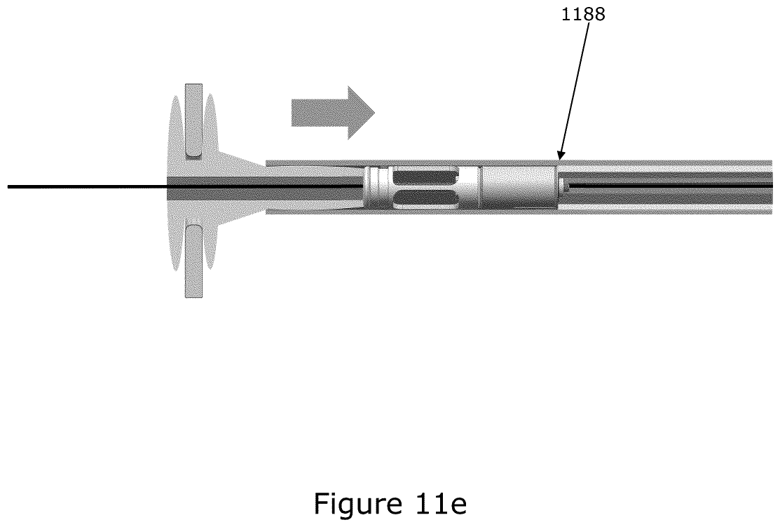

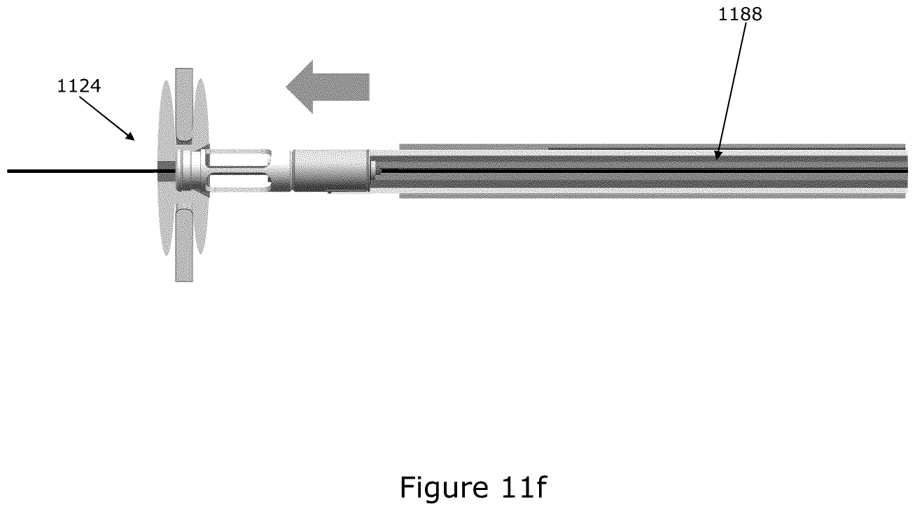

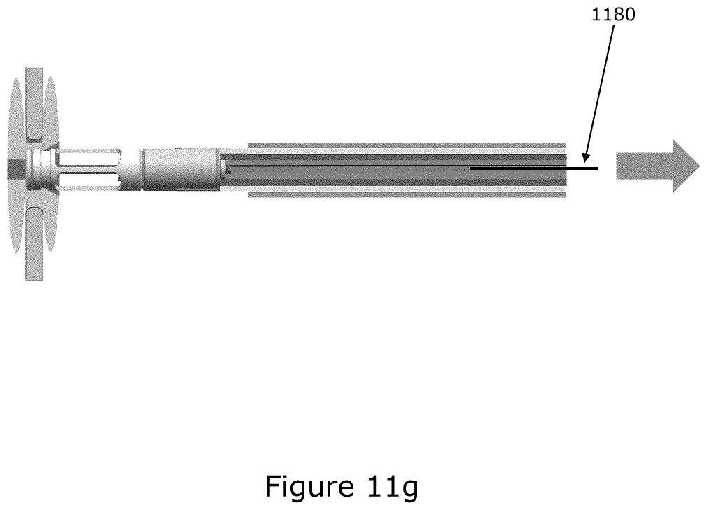

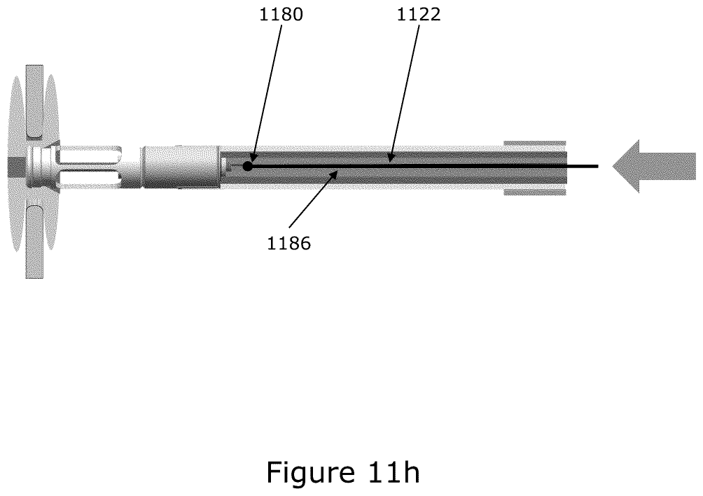

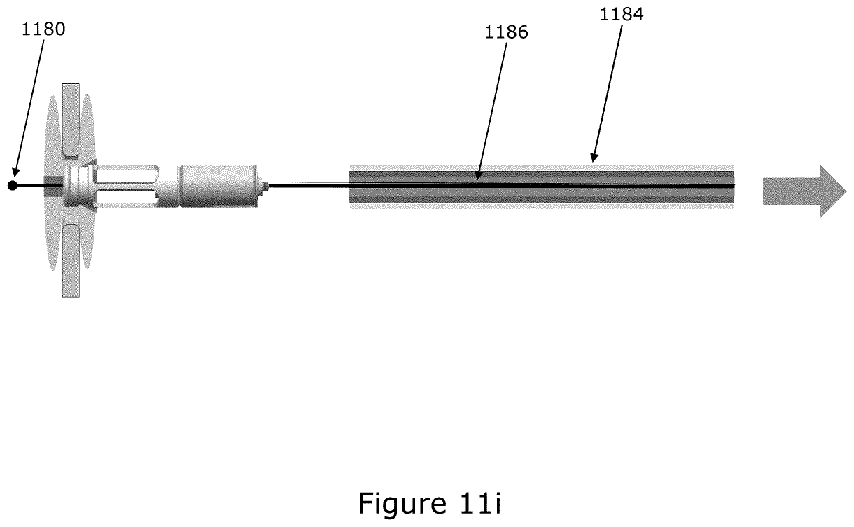

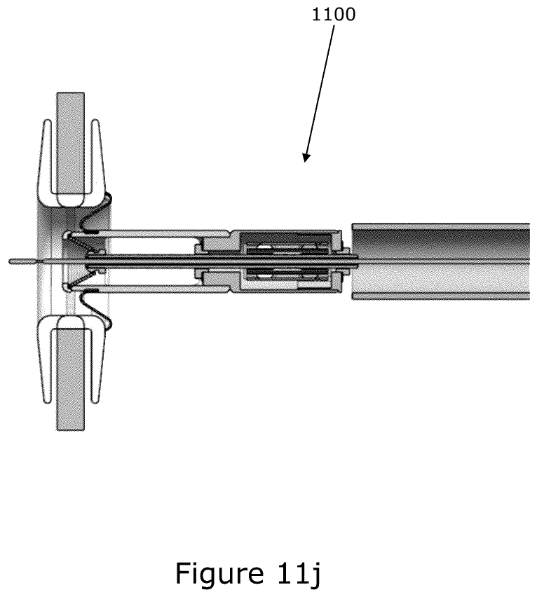

FIGS. 11a-j show an assembly flow for an active shunt apparatus according to an example. For brevity attention is drawn to pertinent features only.

The assembly flow, which may also be termed a delivery flow, proceeds as follows.

As a preliminary step—see FIG. 11a—an active shunt apparatus 1100 is provided. The active shunt apparatus 1100 is as described with reference to the active shunt apparatus illustrated in FIGS. 3-5d. Unless stated otherwise, this does not limit the applicability of the assembly flow to other forms of active shunt apparatus.

In step 1 (FIG. 11b), a guidewire 1180 is installed and provided through an opening 1124. Techniques common in the field may be used to achieve this step.

In step 2 (FIG. 11c), components of the active shunt apparatus are fed over the guidewire 1180 via a delivery system 1182, e.g., a 14 Fr (approx. 4.667 mm outer diameter) delivery sheath. As a result of this step at least a portion of the active shunt apparatus that comprises the left atrium side of the fixture (left atrium side of the shunt) extends through the opening 1124.

An inner portion of the delivery system, e.g., an inner pusher sheath 1184, may be attached to the components of the active shunt apparatus that function as a valve. A wire and valve power line sheath 1186 may be threaded onto a thread rod that connects to the piston of the active shunt apparatus.

In step 3 (FIG. 11d), an outer sheath 1188 of the delivery system 1182 is removed to release the left atrium side of the fixture.

In step 4 (FIG. 11e), the outer sheath 1188 is removed further to release the right atrium side of the fixture.

In step 5 (FIG. 11f), the wire and valve power line sheath 1186 is used to advance the valve to block the opening 1124.

In step 6 (FIG. 11g), the guidewire 1180 is removed.

In step 7 (FIG. 11h), a pressure sensor 1108 and electrical wire 1122 are advanced through the wire and valve power line sheath 1186.

In step 8 (FIG. 11i), the pressure sensor 1108 is located is the left atrium and the inner pusher sheath 1184 and the wire and valve power line sheath 1186 are removed.

The installed arrangement of the active shunt apparatus 1100 is shown in FIG. 11j.

According to another example, an active shunt apparatus as described above is implanted via a transvenous procedure and consists of two separate parts: 1) the deformable valve system (to fix on the septum) connected to a cable (that runs from the deformable valve to the patient's skin surface (preferably below the collarbone on the left side of the chest where an incision is made to access the subclavian vein); and 2) a control box that is attached to the cable end and implanted subcutaneously. The whole valve-cable part is placed inside a splitable guide tube.

Prior to the delivery of the active shunt apparatus, a puncture is created on the septum via standard procedures (e.g., by needle or RF ablation catheters). The active shunt apparatus is delivered from the incision below the collar bone; and a needle is placed into the subclavian vein, through which a guide wire is inserted and pushed to the position, passing the puncture created on the septum. The end of the guide wire (outside of the patient) is threaded through the valve tip (that is collapsed and is just at the end of the splitable tube, designed to accept the guide wire coaxially). The tube, containing the valve-cable part, is then pushed over the wire to pass the septum and enter the left atrium.

At this point in the delivery, the outside end of the cable is held and the tube is retracted slowly to release the distal nitinol (mounting) flange that opens into the left atrium on the septum. The tube is then retracted further to release the second (mounting) flange into the right atrium. Further retraction of the tube results in releasing the deforming occluder (e.g., the tube-spiral valve). The tube is then retracted and removed completely. A splitable tube is preferred for this example, since the end of the cable outside of the patient is terminated with a connector (to be screwed on or clamped on the control box) whose diameter is larger than the tube. Once the tube is completely removed, the cable end is connected to the control box that is implanted subcutaneously and the incision is closed.

Also disclosed is a computer program comprising computer code configured to perform the method shown in FIG. 10. The computer program may comprise one or more computational algorithms. The computational algorithms may comprise one or more of a proportional-integral-derivative controller, a (neuro) fuzzy controller, an expert system and an artificial intelligence-based controller.

### REFERENCES

1. Feldman et al, “Transcatheter Interatrial Shunt Device for the Treatment of Heart Failure Rationale and Design of the Randomized Trial to REDUCE Elevated Left Atrial Pressure in Heart Failure (REDUCE LAP-HF I)”, Circ Heart Fail. 2016;9:e003025. DOI: 10.1161/CIRCHEARTFAILURE.116.003025.
2. Feldman et al, “Transcatheter Interatrial Shunt Device for the Treatment of Heart Failure With Preserved Ejection Fraction (REDUCE LAP-HF I [Reduce Elevated Left Atrial Pressure in Patients With Heart Failure]) A Phase 2, Randomized, Sham-Controlled Trial; Circulation. 2018;137:364-375. DOI: 10.1161/CIRCULATIONAHA.117.032094.
3. Nashat H1, et al, “Atrial septal defects and pulmonary arterial hypertension”, J Thorac Dis. 2018 Sep;10(Suppl 24):S2953-S2965. doi: 10.21037/jtd.2018.08.92.
4. Vijarnsorn C, et al, “Contemporary survival of patients with pulmonary arterial hypertension and congenital systemic to pulmonary shunts”, PLoS One. 2018 Apr. 17;13(4): e0195092. doi: 10.1371/journal.pone.0195092.eCollection 2018.

The applicant hereby discloses in isolation each individual feature described herein and any combination of two or more such features, to the extent that such features or combinations are capable of being carried out based on the present specification as a whole, in the light of the common general knowledge of a person skilled in the art, irrespective of whether such features or combinations of features solve any problems disclosed herein, and without limitation to the scope of the claims. The applicant indicates that the disclosed aspects/embodiments may consist of any such individual feature or combination of features. In view of the foregoing description, it will be evident to a person skilled in the art that various modifications may be made within the scope of the disclosure.

1. An active shunt apparatus comprising:
a fixture attachable to a septum of a patient's heart to form a passage between left and right chambers of the patient's heart;
a reversibly deformable occluder configured to control a flow of blood through the passage between the left and right chambers;
at least one pressure sensor configured to measure a blood pressure associated with one or more portions of the patient's cardiovascular system; and
a control unit configured to, based on a signal received from the at least one pressure sensor, cause deformation of the occluder and a corresponding change in the blood pressure.

2. The active shunt apparatus of claim 1, wherein the control unit is configured to cause deformation of the occluder when the measured blood pressure is above or below a predefined pressure threshold.

3. The active shunt apparatus of claim 2, wherein the control unit is configured to cause deformation of the occluder from a first state to a second state to increase the flow of blood from the left chamber to the right chamber when one or more of the left atrial filling pressure, left-right atrial differential pressure, left ventricular filling pressure and pulmonary artery pressure is above the predefined pressure threshold.

4. The active shunt apparatus of claim 3, wherein the control unit is configured to generate a signal to notify a user of the active shunt apparatus that deformation of the occluder from the first state to the second state has occurred.

5. The active shunt apparatus of claim 1, wherein the control unit is configured to cause deformation of the occluder to maintain the blood pressure within a predefined pressure range.

6. The active shunt apparatus of claim 5, wherein the occluder is configured to undergo continuously variable deformation, from anywhere between a first state allowing a minimum rate of blood flow through the passage and a second state allowing a maximum rate of blood flow through the passage, to maintain the blood pressure within the predefined pressure range.

7. The active shunt apparatus of claim 6, wherein the occluder is configured such that the minimum rate of blood flow through the passage is non-zero.

8. The active shunt apparatus of claim 1, wherein the control unit is configured to further cause periodic deformation of the occluder to reduce tissue formation within the passage or endothelialisation of the occluder.

9. The active shunt apparatus of claim 1, wherein the control unit comprises an actuator connectable to the occluder by a cable, and wherein the actuator is configured to pull, push or release the cable in response to the signal received from the at least one pressure sensor to cause deformation of the occluder.

10. The active shunt apparatus of claim 9, wherein the actuator comprises one or more of an electromechanical system, an electromagnetic system, a microgear system, a microwinch, a motor, a pair of rollers, and a piezoelectric drive mechanism.

11. The active shunt apparatus of claim 9, wherein the fixture comprises distal and proximal flange portions configured to contact respective left and right walls of the septum when attached, and wherein one end of the occluder is attached to the cable such that the occluder abuts the proximal flange portion of the fixture to inhibit the flow of blood when the cable is pushed or released.

12. The active shunt apparatus of claim 9, wherein the cable comprises an inner tube axially movable within a stationary outer tube, and wherein one end of the outer tube is attached to a housing of the actuator and the other end of the outer tube is attached to the distal flange portion of the fixture.

13. The active shunt apparatus of claim 12, wherein the active shunt apparatus further comprises a rigid tube having first and second ends, the first end of the rigid tube attached to a first end of the occluder and the second end of the rigid tube attached to the inner tube of the cable.

14. The active shunt apparatus of claim 13, wherein a second end of the occluder is attached to the inner tube of the cable via a slider configured to slide co-axially within the rigid tube.

15. The active shunt apparatus of claim 12, wherein the inner tube of the cable comprises an electrically conductive material and is configured to form an electrical connector of the at least one pressure sensor.

16. The active shunt apparatus of claim 9, wherein the active shunt apparatus further comprises a piston housed within a cylinder, a first end of the piston attached to the occluder and a second end of the piston attached to the cable.

17. The active shunt apparatus of claim 16, wherein the first end of the piston is attached to the occluder via a bearing configured to inhibit rotational motion of the cylinder.

18. The active shunt apparatus of claim 16, wherein the cylinder is attached to the fixture via one or more collapsible arms.

19. The active shunt apparatus of claim 1, wherein the occluder is attached to, or formed as an extension of, the fixture.

20. The active shunt apparatus of claim 1, wherein the fixture and occluder are sufficiently collapsible to enable endovascular delivery thereof via a catheter.

21. The active shunt apparatus of claim 20, wherein the fixture has a collapsible wire mesh structure and the occluder has a collapsible wire mesh structure or a collapsible spiral spring structure.

22. The active shunt apparatus of claim 21, wherein the collapsible wire mesh structure or collapsible spiral spring structure is formed from a super-elastic material.

23. The active shunt apparatus of claim 22, wherein the super-elastic material comprises a shape memory alloy such as nickel titanium.

24. The active shunt apparatus of claim 21, wherein at least part of the collapsible wire mesh structure or collapsible spiral spring structure comprises a polymer coating or membrane.

25. The active shunt apparatus of claim 24, wherein the polymer coating or membrane is formed from one or more of polyurethane, silicone and polyethylene terephthalate, optionally impregnated with an eluting agent.
